(19) United States
(12) Patent Application Publication
(10) Pub. No.: US 2025/0090454 A1
(43) Pub. Date: Mar. 20, 2025

(54) INNOVATIVE DELIVERY SYSTEM DEVICE AND METHOD FOR PHARMACEUTICAL IMPLANTATION FOR TIMED RELEASE

(71) Applicants: Robert G McKenzie, JR., Tarpon Springs, FL (US); Jacob J Beckel, Tampa, FL (US)

(72) Inventors: Robert G McKenzie, JR., Tarpon Springs, FL (US); Jacob J Beckel, Tampa, FL (US)

(73) Assignee: CEMSTAR, LLC, Tarpon Springs, FL (US)

(21) Appl. No.: 18/770,554

(22) Filed: Jul. 11, 2024

Related U.S. Application Data
(63) Continuation-in-part of application No. 18/370,457, filed on Sep. 20, 2023.
(63) Continuation-in-part of application No. 18/371,995, filed on Sep. 22, 2023.

(51) Int. Cl. A61K 9/00; A61K 31/568; A61K 38/26; A61K 45/06; A61M 37/00

(52) U.S. Cl. CPC A61K 9/0024; A61K 31/568; A61K 38/26; A61K 45/06; A61M 37/0069

(57) ABSTRACT

The invention is a delivery system device and method for implantation of pharmaceuticals, such as hormones, including testosterone for military forces to increase stamina and aid in wound recovery, for timed release into a patient to be determined by a medical professional. This timed-release device will consist of an axially containing and longitudinally flexible elongated member having an axial wall of varying material, that wall consisting of a polymer dissolving according to a pre-determined schedule regulated by the dissolution rate of the polymer or polymer blend. The delivery system and method further prevents the extrusion or migration of the pellets from the site of implantation and allows for easier removal after the time of implant if necessary.

### CROSS-REFERENCE TO RELATED APPLICATIONS

This application claims benefit of U.S. Provisional Application 63/409,012 filed on Sep. 22, 2022, U.S. non-provisional applications Ser. Nos. 18/370,457 filed Sep. 20, 2023 and 18/371,995 filed Sep. 22, 2023 and priority to those dates.

### BACKGROUND OF THE INVENTION

### Field of Invention

The present invention relates to systems and methods for delivering pharmaceutical ingredients to treat animals and humans in a timed released manner and to prevent the migration of the pharmaceutical from the treatment site.

The invention is a delivery system device and method for implantation of pharmaceuticals, such as hormones, including testosterone for military forces to increase stamina and aid in wound recovery, for timed release into a patient to be determined by a medical professional. A strand is implanted containing one or more sleeves with varying dissolution rates of the sleeve, and the sleeve contains the desired pharmaceutical to be dissolving according to a pre-determined schedule. This timed-release device will consist of an axially containing and longitudinally flexible elongated member referred to as a strand or elongated member strand composed of sleeves having an axial wall of varying material, said sleeve wall consisting of a polymer dissolving according to a pre-determined schedule regulated by the dissolution rate of the polymer or polymer blend. The delivery system and method further prevents the extrusion or migration of the pellets from the site of implantation and allows for easier removal after the time of implant.

### Background

In hormonal therapy, one method for treating low testosterone is to make a series of implants made from testosterone in one of its many salts, i.e., Testosterone, including testosterone cypionate, compressed into a pellet with a binding agent that releases over time, such as stearic acid. These pellets typically dissolve over the course of about 3 months making the active ingredient, Testosterone, available to the body and thereby increasing Testosterone levels in the patient. These loose pellets are dependent on the tissue in the body to hold the implant in place. These pellets can be purchased from several sources and/or compounded. Patient complaints are common because of the pellets being extruded from the injection site or the pellets migrating to other areas of the body.

The limitation in the current art of the pellet implantation device and method is that the device and method are dependent on the compression forces to make the pellet and the binding agent (such as stearic acid) break down over time in the body to release the active pharmaceutical ingredient, such as testosterone or one of its salt forms. This typically is about 3 months thereby requiring the patient to return for additional implantation. It is relatively impractical if not impossible to have a longer horizon than three months for a single pellet with a consistent pattern of release by that pellet under current technology. Even though a local anesthetic is used for example, for quarterly implantation, there is trauma at the injection site from the pellet implant and the area is sore which is uncomfortable for the patient leading to non-compliance for return visits over time.

No prior art proposes to use a trocar to insert one or more bioabsorbable sleeves calibrated to enable a pharmaceutical to be absorbed over a particular time containing one or more pellets to be absorbed upon the sleeve degradation.

In type two diabetes, pre-diabetes or weight loss, a class of drugs are currently being used that require frequent injections. This invention proposes a delivery system that can be made into a surgical implant that can be implanted every 3, 6 or 12 months. This class of drugs is the GLP-1 agonist. They include Semaglutide, examples are Ozempic and Wegovy. Dulaglutide such as Trulicity. Liraglutide such as Victoza and Saxenda. Extenatide such as Byetta and Bydureion, All requiring weekly or more often injections. The GLP-1/GIP agonist includes Tirzepatide trade name Mounjano which is also a weekly injection. These weekly or more often injections can be difficult for patient compliance. Many patients are not comfortable with self injection and going to a doctors office once a week is not sustainable. As soon as one stops this therapy weight gain or diabetic symptoms return. An implant with slow release can eliminate the compliance issue with the patient when the physician implants a new pellet or stranded pellets at regular intervals such as 3, 6 or 12 months.

No prior art proposes to use a trocar to insert one or more bioabsorbable sleeves calibrated to implant and enable a GLP receptor agonist, whether of the weekly injection type or the daily injection type to be administered to a patient.

Electrospinning, mixing a polymer with a drug in a beaker, can create nanofibers with the drug acting like a bead on a string of the polymer but its short one to two day duration in the body offers no advantage to the current method of compressed pellets dissolving up to (at least) three (3) months. The polymer sleeve prevents migration of the pellets and adds a predictable breach time allowing the pellets to dissolve at a standard rate as indicated in the package insert or other available literature.

### SUMMARY OF THE INVENTION

The invention is a delivery system device and method for implantation of pharmaceuticals, such as hormones, including testosterone for military forces to increase stamina and aid in wound recovery, for timed release into a patient to be determined by a medical professional. This timed-release device will consist of an axially containing and longitudinally flexible elongated member referred to as a strand or elongated member strand, composed of sleeves having an axial wall of varying material, said sleeve wall consisting of a polymer said wall consisting of a polymer dissolving according to a pre-determined schedule regulated by the dissolution rate of the polymer or polymer blend. The delivery system and method further prevents the extrusion or migration of the pellets from the site of implantation and allows for easier removal after the time of implant.

The present invention relates to systems and methods for delivering pharmaceutical ingredients to treat animals and humans in a timed released manner and to prevent the migration of the pharmaceutical from the treatment site. One or more biocompatible polymer sleeves with hormones or other drug is prepared and then combined together to form a strand, the pharmaceutical to be released at a predetermined window of time, based on the composition of the sleeve, in the body. The strand composed of the sleeves is placed in the body.

An advantage of this invention is that the drug is delivered in much smaller continuous dosages which can minimize some of the serious side effects described in the package insert for the drug.

Chemical and natural compounds can be compressed into pellets using cholesterol, steric acid or other binding agents, then implanted under the skin to release over one to three months with most of the release within one to two months. A polymer sleeve forms a casing—a resistant barrier for the enzymes and body fluids. The thickness, composition or perforation on the sleeve can affect the rate at which the body fluids reach the compounds inside the sleeve when implanted in the body. The advantage of this sleeve is to prevent the migration of compressed pellets of drugs, such as hormones, from migrating to the lungs or other parts of the body or from being expelled. It also extends the release time of these compounds giving better patient compliance. For example, this system would potentially require only one visit to the doctor for surgical implantation per year, instead of four, to maintain the required therapeutic levels in the body.

A typical implantation may be ten 75 mg pellets in a PGA perforated polymer sleeve, ten in a 0.051 mm non-perforated PGA polymer sleeve and ten pellets in a 0.102 mm non perforated PGA polymer sleeve for a total implant of 2250 mg over nine months. To extend the release to 12 months, an additional PGA polymer sleeve with a thickness of 0.204 mm and ten 75 mg pellets could be added.

### OBJECTIVES OF THE INVENTION

Currently Military forces have used implanted hormones in pellets to boost the testosterone levels in its forces for stamina, aggression, and help with wound recovery. The current length of time these pellet implants are active is short: up to 3 months. This invention which contemplates a combination to achieve a longer acting horizon for administration of a pharmaceutical product would allow the use of implantation for longer field deployments preferably on an annual basis.

The invention proposes to have manufactured a sleeve in the nature of a casing or a woven suture-like material, including one available from River Point Medical, Portland, Oregon, made of a bioabsorbable material, and proposes to put in that material pellets with pharmaceutical products and carriers so that the combination will prevent the migration of these pellets and hold them in place either one pellet next to each other or spaced apart from each other with polymer spacers in a manner that enables the pharmaceutical product to be absorbed inside tissues of the body over predictable and pre-determined periods of time. The sleeve with the drug pellets is called a strand and can be sealed at the ends and heat treated to tightly cover the pellets.

By extending the time from a pellet implant every three months to administration of strands by one or more trocars once a year a patient would be more compliant yielding the desired benefits of stamina, aggression, and help with wound recovery for a longer period of time and allowing a health care provider to treat more patients.

By using a polymer sleeve or suture like material for a sleeve, the “strand” can accurately be placed in the body prevent expulsion or migration of loose pellets.

It is further desired that an elongated member with pellets be ridged axially to allow the expulsion of the member while maintaining distance from each pellet with a bioabsorbable spacer maintaining distance between pellets for better dissolution.

This elongated member with pellets being ridged axially would also allow for easy retraction of the pellets to adjust the treatment to reach the desired therapeutic blood levels or terminate treatment by removing the strand of implants.

The spacer material used with the pharmaceutical pellets could be echogenic allowing for visualization of the stand using ultrasound.

The spacer material used with the pharmaceutical pellets could be made with a contrast agent allowing for visualization of the strand using CT or MRI scanning techniques.

A further object of the present invention is to provide a delivery system pharmaceutical therapy that is faster and easier than the present system.

Another objective of the present invention is to cause a minimum of trauma to the tissue.

Yet another objective of the delivery system is to allow a controlled release of the active pharmaceutical ingredient into tissue allowing for accurate placement of the implant.

In another aspect the selected bio-compatible material is selected by polymer blend to accurately breach over time allowing the pharmaceutical pellets to begin to slowing release over time.

In another aspect the selected bio-compatible material is selected and layered or increased wall thickness to accurately breach over time.

A further embodiment is active pharmaceutical ingredient embedded in bio-absorbable polymer.

The one or more polymer “sleeve(s)” composing a “strand” can be perforated to allow for controlled release of the pharmaceutical pellets.

The polymer “strand” containing the pharmaceutical can be sterilized prior to implantation.

For the GLP-1 agonist peptides and GLP-1/GIP agonist peptides commonly referred to as the semaglutide category used in type two diabetes, pre-diabetes or weight loss, the objective would be to insert an implant into the patient to improve compliance with extended treatment schedules.

### DETAILED DESCRIPTION OF THE INVENTION

A preferred and demonstrative mode of the invention is given here, but the principles articulated would be evident to a person of reasonable skill in the pharmaceutical arts and in the preparation of strands in accord with this invention. The invention and claims are focused on a device referred to as a strand composed of one or more sleeves for extended release of pharmaceutical products, not the particular pharmaceutical preparation contained in the device.

Chemical and natural compounds can be compressed into pellets using cholesterol, steric acid or other binding agents, then implanted under the skin to release over one to three months with most of the release within one to two months. A polymer sleeve forms a casing—a resistant barrier for the enzymes and body fluids. The thickness, composition or perforation on the sleeve can affect the rate at which the body fluids reach the compounds inside the sleeve when implanted in the body. The advantage of this sleeve is to prevent the migration of compressed pellets of drugs, such as hormones, from migrating to the lungs or other parts of the body or from being expelled. It also extends the release time of these compounds giving better patient compliance. For example, requiring one visit to the doctor for surgical implantation per year, instead of four, to maintain the required therapeutic levels in the body.

The FDA-approved testosterone pellet, Testopel, contains 75 mg of testosterone, 0.97 mg of steric acid and 2 mg of polyvinylpyrrolidone. It is compressed in pellet form with a diameter of 3.2 mm and a length of 9 mm. Ten pellets, for example (750 mg), would be inserted in a polymer sleeve with a wall thickness of 0.051. Each end of the sleeve would be sealed using a heat source resulting in a 92 mm strand. By varying the sleeve thickness or type of polymer it will determine the dissolution rate in the body. Using a Polyglycolic acid (PGA) 0.051 mm polymer sleeve the dissolution rate could extend a Testopel pellet train by three months, or more (in a non-perforated sleeve). Normally Testopel pellets dissolve in three months. This dissolution rate could be extended by an additional three months by using the polymer sleeve before requiring another implant. The range of testosterone in a healthy man is typically between 300-1000 nanograms per deciliter (ng/dL) and females 15-80 nanograms per deciliter (ng/dL). The implantation of Testopel pellets in humans is a standard procedure and can be implanted in a physician's office using a local anesthetic. There is also pharmacy compounded compressed pellets using steric acid or cholesterol as a binder in various sizes, usually up to 200 mg and can be put into a polymer sleeve to make a strand with the ends sealed to extended to extend the dissolution rate. To have the pellets exposed to the body fluid immediately the polymer sleeve can be perforated for a normal release-one to three months. Yet, the perforated sleeve encapsulates the pellets to prevent migration in or out of the body, which can happen with loose pellets alone.

A typical implantation may be ten 75 mg pellets in a PGA perforated polymer sleeve, ten in a 0.051 mm non-perforated PGA polymer sleeve and ten pellets in a 0.102 mm non perforated PGA polymer sleeve for a total implant of 2250 mg over nine months. To extend the release to 12 months an additional PGA polymer sleeve with a thickness of 0.204 mm and ten 75 mg pellets could be added.

As an alternative to varying s thickness, sleeve other polymers may be used to extend the release rate of the testosterone pellets. These polymers and blends of polymers are found in sutures used to close wounds in patients. A 90% Polylactide and 10% Poly Glycolide sleeve would provide a barrier for nine months until the sleeve is breached. Then the Testopel or compounded pellet would be released over the next three months for a total of 12 months. Table 1, on page 10, lists the various polymers used in medicine for sutures and also used in polymer sleeves for holding radioactive seeds, preventing migration of the radioactive seeds in the body.

This 12-month release of testosterone is ideal for military use where field implantation is not always available. Military use of testosterone helps speed up wound healing, increases endurance and muscle mass. The loose compressed pellets have been used for military applications. This new and novel method reduces the need for multiple surgical procedures. Generally, only one implantation annually would be required.

### Modes of Invention Applicable to Use of Prior Pharmaceutical Preparation

In accordance with one embodiment of the invention, it is proposed that four strands be implanted as follows. The active pharmaceutical ingredients can be as previously referenced, but the preferred embodiment contains testosterone or a salt Aa
1. Strand #1 contains PGA 100% perforated with holes that allow enzymes to break down the steric acid/testosterone pellet 0-3 month delivery.
2. Strand #2 contains PGA 80% and PLA 20% this dissolves in 3 months at which time the steric acid/testosterone pellets will dissolve over 3 months giving a patient 3-6 months of delivery.
3. Strand #3 contains PGA 50% and PLA 50% this dissolves in 6 months at which time the steric acid pellets will dissolve over 3 months giving a patient 6-9 months of delivery.
4 Strand #4 contains PGA 10% and PLA 90% (or 100% Polydioxanone (PDO) that dissolves in 9 months then steric acid pellets will dissolve for the last 3 months giving the patient 9-12 months of delivery.

In another embodiment, a longitudinally flexible elongated member sleeve with at least partial ridges arranged substantially axially made of material which is bio-absorbable in living tissue is provided for insertion into the body, for example muscle tissue, for sustained release of pharmaceutical ingredients. Pharmaceutical products means pharmaceutical ingredients which terms mean and include FDA approved drugs, drugs compounded by a pharmacist, and API's (Active Pharmaceutical Ingredients) or other pharmacy related items. A plurality of pharmaceutical ingredients in the form of pellets can be encapsulated and positioned in a predetermined array in the member in the desired spaced relationship.

Those pharmaceutical products and ingredients can include any pharmaceutical agent, including diet supplements with mammalian effect, including LDN (low dose naltrexone), disulfram and others such as pain medications optimized for timed release.

The substantially axially, semi-ridged elongated member may be made of any natural and/or synthetic bio-compatible and bio-absorbable materials. Natural and synthetic polymers and copolymers can be used. Examples of synthetic bio-absorbable polymer materials are polyglycolide and polylactide, polydioxanone and polymeric materials and their equivalents. Such polymeric materials and their equivalents are more fully described in U.S. Pat. Nos. 3,565,869, 3,636,956, 4,052,988 and European Patent application 30822. The materials in this paragraph are referenced generally as “bioabsorbable polymeric materials.”

When the term stearic acid is used, its meaning is intended to cover all pharmaceutical carriers known in the art that are formulated with a particular pharmaceutical to both have the pharmaceutical carrier dissolve at a pre-determined rate and the pharmaceutical be absorbed at a pre-determined rate. The strand will have the purpose of keeping the pellets in one location in the body so that the pellets do not migrate pending dissolution and absorption of the pharmaceutical in the pellet.

The hormone testosterone or one of its salts can be pressed into a pellet with stearic acid, cholesterol or other binding material to give a pellet that is consistently released over 3 months. A sleeve would be closed off at one end, preferably with a heat source, and the pellets loaded into the sleeve by themselves or with polymer spacers in between them, then sealed at the other end, preferably with a heat source. The heat source can be an iron, or soldering iron or heat sealer. Other sealing methods such as glue can be used but must be body soluble and non-toxic. The sleeves could be made of the following materials to give the desired dissolution times post sterilization. The materials in this invention are available from, among other sources, Corbion Biotech, Inc. in Lenexa, Kansas and Sigma-Aldrich of St. Louis, Missouri.

TABLE 1

| | | Testosterone | |
|---|---|---|---|
| Material | Release Time | Release | |
| 100% Glycolide | No delay in the breakdown | 0-3 | months |
| Perforated | of the pellet | | |
| 80% Polyglycolic Acid | 3 month until the strand | 3-6 | months |
| (PGA) & 20% Polylactide | is breached | | |
| Acid (PLA) | | | |
| 100% Polydioxanone | 6 months until the strand | 6-9 | months |
| (PDO) | is breached | | |
| 90% Lactide & 10% | 9 months until the strand | 9-12 | months |
| Glycolide | is breached | | |

This is an example of the materials and not all inclusive of materials the invention can utilize.

Another way to vary the above times is to vary the thickness of the polymer wall composing the sleeve and in turn the strand. In general, a thickness of 0.05 mm is contemplated but it could be 0.2 mm. The key is that the inside diameter of the sleeve must be larger than the outside diameter of the pellet. A typical outside diameter of the pellets is 3.0 mm, 3.2 mm, 4.3 mm, 4.5 mm and 5.62 mm in diameter. For women, the smaller sizes of 3.0-3.2 mm would be typical and for men, the larger sizes would be typical. The length of the pellets within the sleeve would be typically 9 mm and would be cylindrical. The typical sleeve would have 10-12 pellets and the preferable implant of the strands would be four sleeves with the varying release referenced in Table 1. The strand can be shrink-wrapped over the pellet. Normally one size of pellet would be used. For variations in body weight, more or less pellets would be loaded in the strand. Other ways to vary the dissolution rates, in addition to varying wall thickness, would be to vary the polymer ratio, encase one strand within another with the inside strand having an inside diameter larger than the outer diameter of the pellet, and varying the texture of the strand as woven versus smooth.

A manufacturer of strands suitable for the contemplated invention are available from Theragenics Corporation of Buford, Georgia. Alternatively, product is also available from River Point Medical based in Portland, Oregon.

In between the encapsulated pellets, there can be placed a bioabsorbable spacer made with air bubbles or contrast materials commonly used in the body to illuminate contrast (such as gold seeds or spacers) to allow for visualization via ultrasound, CT, or MRI scans. This can show placement of the stranded material in the body and dissolution of the strand over time. The spacer can cooperate with the strand and the pellets in the time release of pharmaceutical material.

In this application, the spacers can be used in the sleeve which is heat-shrunk over the pellets and spacers. This will allow the health care practitioner to cut in the middle of the spacer to obtain the number of pellets desired. For example, if the pellets can be 10 to a sleeve or a strand and if the patient needs 22 pellets, then a health care practitioner can insert two sleeves or strands of 10 and cut two pellets off the third sleeve or strand, all of which would be placed in the trocar for insertion. The sleeve melts over pellets and the spacers. It fuses with the spacer so the integrity is maintained when the sleeve or strand s cut. This simplifies manufacturing by having the sleeves or trands all one length and later to be custom tailored to the patients dose.

A pre-sterilized kit containing sterile strands with pharmaceutical pellets and a sterile trocar will save the health care provider time. The trocar is used to insert the strand and then retracted leaving the strand in its desired location within the body. The technology for such insertion, leaving and retraction is standard in brachytherapy used for implantation of radioactive seeds. The strands can be sterilized with gamma sterilization or e-beam sterilization or other known methods.

The small diameter of the strands and reduction to one application per year will reduce the trauma to patient and encourage compliance with the therapy.

The timed released of the pharmaceutical pellets can also be achieved as follows.

The first polymer sleeve preloaded or strand post loaded will have perforations allowing the enzymes in the tissue to enter and dissolve the pellets pressed with stearic acid over a period of 3 months. The second sleeve will have a polymer ratio of Polylactide Acid (PLA) to Polyglycolic Acid (PGA) so that that second portion of the strand will dissolve over 3 months during the period of three months to six months after the initial implantation of the strand in a patient during which period the pharmaceutical pellets will be absorbed resulting in an additional 3 months of release to reach 6 total months of therapy. The third strand will have a polymer of Polydioxanone in the sleeve so that the strand will dissolve over 3 months during the period of six months to nine months after the initial implantation of the strand in a patient during which period the pharmaceutical pellets will be absorbed resulting in an additional 3 months of release to reach 9 total months of therapy. The fourth strand will have a polymer ratio of Polylactide Acid (PLA) to Polyglycolic Acid (PGA) so that that fourth strand will dissolve over 3 months during the period of nine months to twelve months after the initial implantation of the strand in a patient during which period the pharmaceutical pellets will be absorbed resulting in an additional 3 months of release to reach 12 total months of therapy. In sum, with additional implantation under this enablement, the device will achieve a total of 12 months of active pharmaceutical ingredient release by the combination of the selection of sleeve material and time-release pellet containing at least one pharmaceutical. As stated, one contemplated enablement is the release of testosterone hormone over the course of one year. As a practical matter, multiple strands are easier to work with, but in theory there could be one long strand of one or more polymers. As an alternative to the above use of various polymers one could use a single polymer and adjust the thickness of the strand to affect the dissolution rate. For example using a Polyglycolic Acid (PGA) sleeve with a wall thickness of 0.002 inches (0.051 mm) perforated for the release of the compressed testosterone pellet in steric acid to give a 3 months dissolution rate, a 0.002 inch (0.051 mm) non-perforated strand for the release of the compress testosterone pellets for 6 months dissolution rate, a 0.004 inch (0.102 mm) non perforated strand for 9 months dissolution rate and a 0.006 inch (0.152 mm) non perforated strand for 12 month dissolution rate. By keeping the polymer material the same, one can reduce the expense of manufacturing the invention.

### Other Modes of the Invention

Besides Polylactic Acid (PLA) or Polyglycolic Acid (PGA), co-polymer or self-reinforcing composites can be used. By using one or more combinations an absorption time can be extended past 5 years.

TABLE 2

| | Melting | Glass- | Modulus | Degradation |
|---|---|---|---|---|
| | Point | Transition | (Gpa) | Time |
| Polymer | (° C.) | Temp. (° C.) | (gigapascals) | (months) |
| PGA | 225-230 | 35-40 | 7.0 | 6-12 |
| L-PLA | 173-178 | 60-65 | 2.7 | >24 |
| DL-PLA | Amorphous | 55-60 | 1.9 | 12 to 16 |
| PCL | 58-63 | (−65)-(−60) | .4 | >24 |
| PDO | 110 | (−10)-0 | 1.5 | 6-12 |

TABLE 3
Biodegradable polymers, properties and degradation time

| | Ratio of | |
|---|---|---|
| | 1^{st} | |
| | polymer | |
| | to 2^{nd} | |
| | polymer | Degradation |
| | in | Time |
| Polymer combination | copolymer | (months) |
| 1) PGA | PGA to | 6-12 |
| 2) TMC | TMC | |
| TMC = trimethenecarbonate | ratio 85:15 | |
| DLPLG = Poly(D,L-lactide- | Lactide to | 4-5 |
| co-glycolide) | glycolide | |
| DLPLG 75/25 | ratio 75:25 | |
| DLPLG 65/35 | Lactide to | 3-4 |
| | glycolide | |
| | ratio 65:35 | |
| DLPLG 50/50 | Lactide to | 1-2 |
| | glycolide | |
| | ratio 50:50 | |

Other alternatives that can be used are referenced in U.S. Pat. No. 7,942,803 issued May 17, 2011.

TABLE 4

| Polymer | Absorption Time | |
|---|---|---|
| *SR PLLA | >5-6 | years |
| **PLLA | >5 | years |
| ***P(D/L) LA 70/30 | 2-3 | years |
| PLA/PGA (PLGA) 80/20 | 1-2 | years |
| *P(D/L) LA 96/4 | 2 | years |
| *SR PGA | 0.5-1 | years |
| PDS (polydioxanone) | 2 | months |
| PGA | 1-2 | months |
| Perforated PGA | Time Zero-3 | months |

*SR is Structure Reinforced
**PLLA is the L-Isomer of PLA
***(D/L) is the D and L isomer

A variation to the system to obtain the same results is changing the ratios of the polymer blend to layer the same or another polymer blend in the axial sleeve wall to slow the access of body fluids to the pellets by delaying dissolution of the wall and accordingly absorption or the pharmaceutical material in the pellet. Alternatively, one can increase the axial wall thickness by using multiple layers of polymer materials (same or different polymer materials one inside the other) in the wall to achieve the desired delayed release of the pharmaceutical pellets. Alternatively, the pharmaceutical pellets themselves can be designed to be bioabsorbed at different times as the wall is bioabsdorbed.

In implementing the invention, one end of the sleeve is heat sealed. The pellets are placed into the sleeve with or without spacers and the other end of the sleeve is sealed. The sleeve is heated, preferably baked in an oven to shrink the sleeve over the pellets and then the strand also heat-shrunk making the strand that in total has a substantially axial wall, with a semi-ridged and longitudinally flexible elongated member. At this point, the strand can be inserted into another sleeve and the process repeated to give a double wall strand, thereby halving and therefore slowing the dissolution rate. This can also be achieved during the extrusion process of the strand material to double the wall thickness.

Different wall thicknesses of different materials with varying dissolutions rates can be combined so that more nuanced release can be achieved.

Thereafter, the strand with one or more sleeves is inserted in a trocar that encompasses the strand for insertion into a patient. Trocar applicators will need to be sized to the strands so that the trocar will have the proper interior diameter and length for application.

An alternative to the sleeve is to embed the pharmaceutical pellets into the polymer, co-polymer, or structure reinforcement of a strand.

The delivery system and method further prevents the extrusion or migration of the pellets from the site of implantation and allows for easier removal after the time of implant.

Another mode of invention of a desirable class of pharmaceutical suitable for time-release delivery for this invention relates to recent weight-loss pharmaceuticals related to GLP-1 agonist and GLP-1/GIP agonists. In type two diabetes, pre-diabetes or weight loss, a class of drugs are currently being used that require frequent injections can be made into a surgical implant that can be implanted every 3, 6 or 12 months. This class of drugs is the GLP-1 agonist. They include Semaglutide, examples are Ozempic and Wegovy. Other examples are Dulaglutide such as Trulicity, Liraglutide such as Victoza and Saxenda, and Extenatide such as Byetta and Bydureion. All require weekly or more often injections. The GLP-1/GIP agonist includes Tirzepatide trade name Mounjano which is also a weekly injection.

These weekly or more often injections can be difficult for patient compliance. Many patients are not comfortable with self-injection and going to a doctor's office once a week is not sustainable. A problem with these therapies is that when use of them stops and no lifestyle change has occurred, often weight gain or diabetic symptoms return. An implant with slow release can eliminate the compliance issue with the patient when the physician implants a new pellet or stranded pellets at regular intervals such as 3, 6 or 12 months.

Semaglutide is marketed in 0.5 mg, 1 mg and 2 mg injections once per week as Ozempic by Novo Nordisk A/S of Bagsvaerd, Denmark. Novo Nordisk, Inc., which has US marketing rights for Novo Nordisk A/S, has a US location at Plainsboro, New Jersey. Semaglutide is marketed in a 2.4 mg injection once per week as Wegovy in a 2.4 mg injection by Novo Nordisk A/S of Bagsvaerd, Denmark. This is designed to slow stomach emptying so one feels “more full” and should be able to comfortably eat less with fewer hunger pains. Semaglutide is targeted at reducing the craving for food as a way of improving health. These are glucagon-like peptide (GLP) receptor agonist drugs.

This invention can utilize a class of drugs that are targeted at reducing A1C, and blood sugar, both a marker for diabetes and the ability to process blood sugar. Dulaglutide is marketed in 0.25, 0.5, 0.75, 1.5, 30 and 4.5 mg injections once per week as Trulicity by Lilly USA LLC of Indianapolis, Indiana. Its target is to improve blood sugar (glucose) in adults and children 10 years of age and older with type 2 diabetes mellitus. A similar member of this class to reduce A1C and blood sugar is liraglutide marketed as Victoza in 1.2 mg and 1.8 mg once daily injectable by Novo Nordisk A/S of Bagsvaerd, Denmark.

Novo Nordisk A/S of Bagsvaerd, Denmark also markets liraglutide as Saxenda in a 3 mg injectable once daily. It is a GLP-1 receptor agonist.

Another class useful in the invention is extenatide marketed as Byetta, Bydureon and Bydureon BCise which are injectable drug pens sold by AstraZeneca Pharmaceuticals LP of Wilmington, Delaware working with amylin Ohio, LLC of West Chester, Ohio, to control blood sugar in people with Type 2 diabetes. The Byetta pen contains sixty does of 5 micrograms. Bydureon BCise is an extended release once a week injectable suspension of 2 mg in a pen.

A patient on 0.5 mg of semaglutide weekly injection regime would receive the semaglutide in a compressed pellet with a binder such as steric acid or cholesterol and 6 mg of semaglutide compressed into pellet, These pellets would be placed in a polymer sleeve perforated for the first 3 months of dissolution and non-perforated for extended release periods depending on the thickness of the polymer strand.

All of these pharmaceuticals described in relation to weight-loss and the GLP-1 and GLP-1/GIP agonists including semiglutide, dulaglutide, liraglutide, exenatide, tirzapatide and similar type of GLP agonists will be generally referred to as GLP-1 or GLP-1/GIP agonists. They may also be referred to as the semaglutide category used in type two diabetes, pre-diabetes or weight loss,

An advantage of this invention related to the GLP-1 and GLP-1/GIP agonists is that the drug is delivered in much smaller continuous dosages which can minimize some of the serious side effects described in the package insert for the drug.

Anti-spasmodic drugs are well-known. They are used in conjunction with this invention to offset side effects of the GLP-1 and GLP-1/GIP agonists. The anti-spasmodic drugs administered per their package inserts include metoclopramide marketed as Reglan and Metozolv ODT. Metoclopramide is generic and available from Evoke Pharma among other companies. Another anti-spasmodic is Zofran® marketed by Novartis Pharmaceuticals Corporation out of East Hanover, New Jersey.

Either one or both of the GLP-1 and GLP-1/GIP agonists and the anti-spasmodic drugs, if not already in a suspension, could be placed optimally in a pharmaceutically acceptable oil, oleogel, hydrogel, bigel, or suspension (said pharmaceutically acceptable oil, oleogel, hydrogel, bigel, or suspension collectively referred to as a “suspension”) to be placed within the sleeves. These would be known to a reasonably skilled practitioner in the art relating to pharmaceutical carriers. They are discussed in Wroblewska M et al, “Oleogels and Bigels as Topical Drug Carriers for Ketoconazole—Development and In Vitro Characterization, Acta Poloniae Pharmaceutica-Drug Research, Vol. 75 No. 3 pp. 777-786 (Thieme Medical and Scientific Publishers, Mumbai, India 2018), and “Hamed R, et al, Development of hydrogels, oleogels, and bigels as local drug delivery systems for periodontitis, Vol. 44(9), pp. 1488-1497, Drug Dev Ind Pharm. Epub 2018 May 7, (Taylor & Francis Group, London 2018) PMID: 29669437.

The embodiments represented herein are only a few of the many embodiments and modifications that a practitioner reasonably skilled in the art could make or use. The invention is not limited to these embodiments. Alternative embodiments and modifications which would still be encompassed by the invention may be made by those skilled in the art, particularly in light of the foregoing teachings. Therefore, the following claims are intended to cover any alternative embodiments, modifications or equivalents which may be included within the spirit and scope of the invention as claimed.

1. A device for extended release of at least one pharmaceutical product comprising:
an elongated implantable member strand slidable into a trocar for implantation;
said elongated member strand being composed of at least one sleeve;
said at least one sleeve being enabled to contain pellets containing at least one pharmaceutical product;
said at least one sleeve containing pellets having a pre-determined pharmaceutical schedule for dissolution of said at least one pharmaceutical product from said sleeve;
said elongated member strand being bioabsorbable;
each said at least one sleeve being formulated from at least one bioabsorbable polymeric material set to dissolve at a selected time so that by the cooperation of the rate of dissolution of each said polymeric sleeve and the rate of dissolution of the pellets contained within each said polymeric sleeve, a combined pre-determined release schedule of the desired at least one pharmaceutical product is achieved.

2. The device for extended release of said at least one pharmaceutical product set forth in claim 1 wherein said elongated member strand is axially rigid and radially flexible.

3. The device for extended release of said at least one pharmaceutical product set forth in claim 1 wherein said elongated member strand has sufficient radial flexibility to maintain locational accuracy.

4. The device for extended release of said at least one pharmaceutical product set forth in claim 1 where said elongated member strand is longitudinally flexible.

5. The device for extended release of said at least one pharmaceutical product set forth in claim 1 where said elongated member is laterally flexible.

6. The device for extended release of said at least one pharmaceutical product set forth in claim 1 where said sleeve contains pellets positioned at various intervals, said pellets containing any one of testosterone or a salt of testosterone.

7. The device for extended release of said at least one pharmaceutical product set forth in claim 1 where said elongated member contains pellets positioned at various intervals.

8. The device for extended release of said at least one pharmaceutical product set forth in claim 1 where the elongated member bioabsorbable strand is perforated to allow instant access to the pharmaceutical pellets by enzymes and phagocytes in the absorption process in living tissue.

9. The device for extended release of pharmaceutical products set forth in claim 1 where the elongated member bioabsorbable strand is absorbed in living tissue from a range of initial placement to over 60 months.

10. The device for extended release of said at least one pharmaceutical product set forth in claim 1 where the elongated member bioabsorbable strand is absorbed in living tissue from a range of initial placement to one month, to two months, to three months or any number of months up to sixty months.

11. The device for extended release of said at least one pharmaceutical product set forth in claim 1 where the elongated member bioabsorbable strand is absorbed in living tissue beginning at a set point after initial placement over a range of a set period of up to 5 years.

12. The device for extended release of said at least one pharmaceutical product set forth in claim 1 where the elongated member bioabsorbable strand is sealed on both ends preventing products of bodily fluids and cells from dissolving the pellets prematurely.

13. The device for extended release of said at least one pharmaceutical product set forth in claim 1 where the one or more materials constituting the elongated member bioabsorbable strand is selected from the group of synthetic bio-absorbable polymer materials.

14. The device for extended release of pharmaceutical products set forth in claim 1 where the pellets themselves have a time-release characteristic according to a pre-determined pharmaceutical schedule.

15. The device for extended release of said at least one pharmaceutical product set forth in claim 1 where the one or more materials constituting the sleeve is selected from the group of Polylactic Acid (PLA), Polyglycolic Acid (PGA), SR PLLA, PLLA, P (D/L) LA 70/30, PLA/PGA (PLGA) 80/20, P (D/L) LA 96/4, SR PGA, PDO (polydioxanone), PGA, Perforated PGA, or composites or co-polymers of materials in this group.

16. The device set forth in claim 1 wherein the polymeric material is selected in varying amounts and ratios from the group of Glycolide, Lactide, and Polydioxanone.

17. The device for extended release of pharmaceutical products set forth in claim 1 where the elongated member bioabsorbable sleeve is selected from polydioxanone.

18. The device for extended release of pharmaceutical products set forth in claim 1 where the one or more strands containing the at least one pharmaceutical product are selected from structural reinforcement materials.

19. The device for extended release of pharmaceutical products set forth in claim 1 forming a strand where the at least one sleeve contains pharmaceutical pellets containing stearic acid or other binding chemicals.

20. The device for extended release of pharmaceutical products set forth in claim 1 where the elongated member bioabsorbable strand is echogenic.

21. The device for extended release of pharmaceutical products set forth in claim 1 where the elongated member bioabsorbable strand is visible with a CT scanner.

22. The device for extended release of pharmaceutical products set forth in claim 1 where the elongated member bioabsorbable strand is visible with an MRI scanner.

23. The device for extended release of pharmaceutical products set forth in claim 1 where the elongated member bioabsorbable strand can be gamma sterilized.

24. The device for extended release of pharmaceutical products set forth in claim 1 where the elongated member bioabsorbable strand can be electron beam sterilized.

25. The device for extended release of pharmaceutical products set forth in claim 1 where the elongated member bioabsorbable strand can be steam sterilized.

26. The device for extended release of pharmaceutical products set forth in claim 1 where the elongated member bioabsorbable strand can be layered to increase absorption time.

27. The device for extended release of pharmaceutical products set forth in claim 1 where the elongated member bioabsorbable sleeve can be extruded with an increased wall thickness to slow absorption time.

28. The device for extended release of pharmaceutical products set forth in claim 1 wherein said elongated member bioabsorbable strand containing pellets can be provided as a sterile kit containing a trocar for the implantation of the strand into the patient.

29. The device for extended release of pharmaceutical products set forth in claim 1 wherein said elongated member bioabsorbable strand containing pharmaceutical pellets can be used to size the internal diameter of the trocar to reduce trauma to the patient.

30. The device for extended release of pharmaceutical products set forth in claim 1 wherein said elongated member bioabsorbable strand containing pharmaceutical pellets has pellets of varying bioabsorbability within said elongated member.

31. The device for extended release of pharmaceutical products set forth in claim 1 wherein said elongated member bioabsorbable strand contains spacers to enable a health care practitioner to select the length of strand or sleeve and amount of pharmaceutical product being administered.

32. A device for extended release of at least one pharmaceutical product comprising:
an elongated implantable member strand slidable into a trocar for implantation;
said elongated member strand being composed of at least one sleeve;
said at least one sleeve being enabled to contain a suspension containing at least one pharmaceutical product;
said at least one sleeve containing said suspension having a pre-determined pharmaceutical schedule for dissolution of said at least one pharmaceutical product from said sleeve;
said elongated member strand being bioabsorbable;
each said at least one sleeve being formulated from at least one bioabsorbable polymeric material set to dissolve at a selected time so that by the cooperation of the rate of dissolution of each said polymeric sleeve and the suspension contained within each said polymeric sleeve, a combined pre-determined release schedule of the desired at least one pharmaceutical product is achieved.

33. The device for extended release of said at least one pharmaceutical product set forth in claim 31 where one of the at least one pharmaceutical products is selected from the group of GLP-1 or GLP-1/GIP agonists.

34. The device for extended release of said at least one pharmaceutical product set forth in claim 14 where one of the at least one pharmaceutical products is selected from the group of GLP-1 or GLP-1/GIP agonists.

35. The device for extended release of said at least one pharmaceutical product set forth in claim 31 where one of the at least one pharmaceutical products is selected from the group of GLP-1 or GLP-1/GIP agonists and said at least one sleeve contains a drug selected from the group of anti-spasmodic drugs to reduce the side effects of the at least one pharmaceutical product selected from the group of GLP-1 or GLP-1/GIP agonists.

36. The device for extended release of said at least one pharmaceutical product set forth in claim 14 where one of the at least one pharmaceutical products is selected from the group of GLP-1 or GLP-1/GIP agonists and said at least one sleeve contains a drug selected from the group of anti-spasmodic drugs to reduce the side effects of the at least one pharmaceutical product selected from the group of GLP-1 or GLP-1/GIP agonists.
